(19) United States
(12) Patent Application Publication
(10) Pub. No.: US 2012/0156309 A1
(43) Pub. Date: Jun. 21, 2012

(54) Using Aqueous Oxygenation to Improve Animal Health & Wellness

(76) Inventor: C. Edward Eckert, New Kensington, PA (US)

(21) Appl. No.: 13/407,604

(22) Filed: Feb. 28, 2012

Related U.S. Application Data
(63) Continuation-in-part of application No. 13/369,385, filed on Feb. 9, 2012.
Application No. 13/369,385 is a continuation-in-part of application No. 12/660,012, filed on Feb. 19, 2010.
Application No. 12/660,012 is a continuation-in-part of application No. 11/857,556, filed on Sep. 19, 2007, now abandoned.
(60) Provisional application No. 61/574,526, filed on Aug. 4, 2011.

(51) Int. Cl. A61K 33/00; A61P 21/00; A61P 17/00

(52) U.S. Cl. 424/613; 424/600; 424/700

(57) ABSTRACT

Dispersing gas in a liquid provides a mixture of saturated, supersaturated or hypersaturated solution to provide a suspension of bubbles containing gas therein into which animal tissue is immersed for health or wellness treatment. A two-phase mixture is provided with dissolved gas as a suspension of microbubbles. Methods for making, maintaining, and using the two-phase mixture are provided. Gas molecules may be introduced into a liquid at high velocity under elevated pressure to form a supersaturated solution that retains the gas, preferably oxygen, in solution when exposed to ambient conditions.

### RELATED APPLICATIONS

This application is a continuation-in-part of U.S. application Ser. No. 13/369,385, filed on Feb. 9, 2012, which is related to U.S. Provisional Application Ser. No. 61/574,526, filed on Aug. 4, 2011, itself being related to U.S. application Ser. No. 12/660,012, filed on Feb. 19, 2010, which is a continuation-in-part of U.S. application Ser. No. 11/857,556, filed on Sep. 19, 2007. All of the aforementioned disclosures are incorporated by reference in their entireties herein.

### FIELD OF THE INVENTION

The present invention relates to solutions of dissolved gas in liquids, and also to mixtures consisting of a solution of gas, e.g., O₂, and a dispersion of the gas in a liquid, e.g., water. More specifically, this invention relates to an improved animal wellness system and method that uses an oxygen environment chemically comparable or superior to an oxygen chamber (tent) but without the potentially hazardous accumulation of a pure or oxygen enriched atmosphere.

### BACKGROUND OF THE INVENTION

This invention relates to solutions having quantities of dissolved gas, and more particularly to mixtures containing a solution having quantities of gas and a dispersion of ultra-small microbubbles of the gas in suspension within the solution. These microbubbles generally have a diameter of less than about 125 microns, and preferably less than 50 microns, and are defined as such. It is desirable in the context of this invention to minimize the terminal velocity of the microbubbles within the suspending liquid. Since the residence time of a bubble in a liquid is inversely proportional to the square of the bubble diameter, such microbubbles will have an extended contact time with a fluid as compared to a system consisting of larger diameter bubbles. This quality is known as gas holdup. Additionally, the microbubble/liquid interfacial area is inversely proportional to the square of the bubble diameter. Therefore, it will be shown that a solution of a solute gas such as oxygen in a solvent liquid such as water can be manipulated to produce a dispersion of microbubbles consisting of solute gas nucleated and precipitated from the liquid. This results in extended residence time and a high interfacial area that maximizes the contact between the gas (as microbubbles) and a surface placed within the liquid, such as living tissue.

Various means of exposing living human tissue, in particular skin, to oxygen have been used. These include hyperbaric chambers where a pure oxygen atmosphere is pressurized to, typically, under 3 atmospheres of pressure, and oxygen tents that operate at atmospheric pressure. The use of oxygen tents has known benefits. Cutaneous oxygen uptake through the skin surface can be locally and systemically beneficial by augmenting respiration as an oxygen uptake mechanism. Since cutaneous uptake does not depend on blood circulation through capillaries, local oxygen uptake through the extremities can be beneficial in providing tissue health. Further, a generally enhanced disposition and accelerated recovery from physical exertion can result, as reported by users of oxygen tents. This invention will create similar or enhanced conditions to accomplish these benefits without the accumulation of oxygen in a tent environment. It is contemplated that oxygenated solutions and suspensions of oxygen microbubbles thereof can be used in a variety of applications where an enhanced dissolved oxygen content is desired.

In the human and animal medical community, it is generally known that the effect of oxygen on living tissue can be characterized by three regimes, namely, metabolic enhancement (growth accelerator), metabolic inhibition (growth arrest), and toxicity. In the former regime, oxygenated solutions and microbubble suspensions can be used to accelerate the healing and regeneration rate of damaged tissue. When wounds begin to heal in humans, for example, fibroblastic cells divide and spread throughout the wound area. The fibroblastic cells produce collagen, an important protein that facilitates healing. Supplying sufficient quantities of oxygen to the wound area significantly enhances fibroblast proliferation. In particular, the fibroblastic cells use amino acids hydroxylated with oxygen to synthesize collagen chains.

In addition to treating wounds, oxygen can be used in topical applications for cleaning and revitalizing skin. Various surface contaminants and even parasites can be mediated through exposure to elevated levels of molecular oxygen. In a manner much less aggressive than using a free radical form of oxygen, such as hydrogen peroxide, for example, organic oils on animal skin can be oxidized, solubilized, and transported away by the water component of this form of treatment. Mites and other skin parasites afflicting animals may be exposed to toxic oxygen levels inherent with the oxygen microbubble/water dispersion and residual dissolved oxygen.

Recovery time from physical exertion by, for example, a racehorse can be reduced through the ability to impart oxygen directly to muscular tissue in the extremities. The so called “taught” form of hemoglobin that resides within inter-muscular tissue can be oxygenated by direct cutaneous oxygen transport rather than through hematological means. In situations where the animal is exerted above the lactate threshold, cutaneous oxygenation may facilitate a more rapid recovery from anaerobic metabolism. Bruising and other contusions incurred through exertion may respond to this oxygenated environment as well.

In addition, oxygen can revitalize skin cells by joining with protein molecules to nourish the cells and produce collagen. It is even possible that dissolved oxygen and microbubble suspensions can stimulate hair follicles and consequentially hair growth.

The amount of oxygen initially dissolved into solution is largely dependent on the method used to dissolve the oxygen gas into solution. Generally, these methods consist of two steps: creating a solute gas/solvent liquid interfacial area, and, exposing the gas/liquid mixture to elevated pressure. The former step affects the kinetics or rate at which the solution process occurs while the latter determines the maximum theoretical dissolved. Small bubbles create interfacial area and promote more favorable kinetics. The second step is a pressure-concentration relationship, such as Henry's Law for dilute solutions and Sievert's Law for diatomic gases at higher concentrations. These steps may be combined, although the source of oxygen must operate at a higher final pressure rather than allowing a pump, for example, to pressurize both the liquid and gas components after the gas has been introduced.

One common method for oxygenating water is the coarse bubble aeration process, which is a subset of aeration methods known categorically as air diffusion. Pressurized air or oxygen gas is introduced through a submerged pipe having small holes or orifices into a container of water. Gas pressure is sufficient to overcome the hydrostatic head pressure, and also sustains pressure losses during passage through the small gas orifices. As a result, bubble aeration occurs at relatively low pressures; this pressure being predominantly a function of tube immersion depth and density of the liquid, which in this case is water.

Since all interphase interfaces have a characteristic surface energy, the creation of interfacial (surface) area is an energetic process. As a gas passes through an orifice, for example, pressure energy is converted to kinetic energy, which consequently satisfies the energetic requirements of the system for the production of surface area. Area and velocity are inversely proportional; hence, as the orifice diameter decreases, the corresponding pressure drop and gas velocity increase, and more surface area is generated. Smaller bubbles result. This process has a limiting condition, however, in that the amount of heat (as irreversible work) that is produced is inversely proportional to the square of orifice diameter. It therefore becomes impractical and energetically inefficient to operate at exceptionally small orifice diameters. This process also has an absolute limit as a gas velocity of Mach 1 is approached within the pore of a porous medium used to create bubbles. Because a pore lacks the convergent/divergent geometry required to achieve supersonic flow, increasing pressure beyond the critical pressure will not result in a further reduction of bubble size.

Since oxygen therefore is introduced into solution at relatively low pressures in the coarse bubble aeration process, the oxygen bubbles are relatively large. As a result, the aggregate bubble surface area for a dispersion of bubbles produced by bubble aeration is relatively small. The limited surface area produced by bubble aeration limits the rate at which gas can be dissolved and practically limits the concentration of gas that can be dissolved into solution. Oxygen dissolution is a function of the interfacial contact area between gas bubbles and the surrounding medium, and bulk fluid transport (mixing) in the liquid phase. In particular, the rate of oxygen dissolution is directly proportional to the surface area of the bubbles. A dispersion of very small bubbles, e.g., bubbles having diameters in the order of 50 microns, will have a much larger total surface area than a dispersion of large bubbles occupying the same volume. Consequently, the rate of oxygen dissolution in bubbling aeration is limited by the size of the bubbles introduced into the solvent. Fluid mixing is also very limited in bubbling aeration because the only energy source available for agitation is the isothermal expansion energy of oxygen as it rises in the solution.

Oxygen dissolution in bubbling aeration is also limited by ambient pressure conditions above the solution. If the solution being aerated is exposed to atmospheric conditions, the dissolved oxygen concentration will be limited to the solubility limit of oxygen (at its partial pressure in air of 0.21 atm.) under such conditions. The desirability of bubbling aeration is further hampered by equipment and energy requirements. Large blower units are used to force the gas bubbles into the carrying liquid. These blowers generate high-energy costs and often require special soundproof installations or other engineering costs. Bubble aeration is therefore an impractical process for producing oxygenated solutions or solution/suspensions for health related applications.

Other methodologies have been used to prepare oxygenated solutions based on pressure tanks and adaptations of carbonator devices that dissolve carbon dioxide in water. For a given pressure and temperature, the solubility of carbon dioxide in water exceeds that of oxygen by over an order of magnitude. Carbonators therefore are acceptable for preparing carbonated water solutions but not oxygenated solutions.

In this invention, conditions most favorable to produce a dispersion of small diameter microbubbles in a suspending solvent liquid with high interfacial area are created either at an elevated pressure or with a subsequent increase of pressure. The elevated pressure environment will dissolve the gas in the liquid, since the concentration of a gas in solution and the pressure over the solution is directly related.

High interfacial area enhances the kinetics of the dissolution process, and pressure establishes the maximum concentration of the gas held in solution. For wellness applications, oxygen will be intentionally nucleated from an aqueous oxygen solution to form a dispersion of microbubbles held within the solution and capable of providing the benefits of pure oxygen. Living tissue, in particular, animal skin and/or muscle, that is exposed to such an environment will have the opportunity for oxygen uptake both from the suspension of pure oxygen microbubbles and oxygen adsorbed from the liquid solution at near atmospheric pressure.

### SUMMARY OF THE INVENTION

It is an object of this invention to provide a novel system for dissolving gas in a liquid. It is another object of this invention to provide a novel system for incorporating large quantities of gas in liquids. It is yet another object of this invention to dissolve large quantities of molecular oxygen in water. It is still another object of this invention to provide large quantities of molecular oxygen in water to provide a saturated solution of metastable molecular oxygen water solution and to create a holdup of microbubbles in suspension within the solution. And still it is yet another object of this invention to use said molecular oxygen water suspension-solutions for health and wellness applications for animal applications similar to oxygen tents. These applications include skin conditions, parasites, exertion recovery, and contusions. Finally, it is another object of this invention to produce saturated and hypersaturated gas-liquid solutions that can be stimulated to nucleate gas bubbles and resulting dispersions for the purposes of providing benefits for animal skin and/or muscle tissue.

These and other objects will become apparent from the claims and drawings appended hereto. In accordance with these objects, there is provided a method of dispersing a gas in a liquid to provide a mixture of a saturated, supersaturated or hypersaturated solution of the gas in the liquid and to optionally provide a suspension of bubbles containing the gas therein. The method comprises the steps of providing a liquid pumping means, a liquid for introducing to the liquid pumping means, and pumping the liquid to a pressure greater than 0.8 atmospheres gage pressure to provide a pressurized liquid. A gas is introduced to the pressurized liquid and dispersed therein to provide a solution having the said gas dissolved therein and having bubbles of the gas. The solution is then subjected to a shearing action to reduce the size of the bubbles to provide a highly hypersaturated liquid and bubbles of said gas having a diameter as low as 5 μm.

Based on the foregoing, an oxygenated mixture is provided having dissolved molecular oxygen content well above the equilibrium limit at ambient conditions. The oxygenated mixture can supply a large amount of molecular oxygen in a medium that is not traumatic to skin tissue. Since the dissolution of oxygen into solution occurs under hyperbaric conditions, a large concentration of oxygen is dissolved into solution. The resulting solution can have a dissolved oxygen content as high as 2000 mg/l. In one embodiment of the solution, an oxygen-enriched solution is accompanied by a dispersion of micro-bubbles held in suspension.

A method for using the oxygenated solution and dispersion for animal health and wellness benefits is provided. The method includes the step of filling a tank or bath with oxygenated solution. The beast body being exposed can be locally, regionally or almost fully immersed in said bath so that areas of the body are submerged into the oxygenated solution and dispersion for contacting skin. As this solution circulates about the tissue layers, the dissolved oxygen nucleates into fine bubbles that attach to skin fragments. Additional energy, as produced by ultrasonic means, can be imparted to facilitate the nucleation of gas bubbles. A volume change occurs upon nucleation of the oxygen bubbles. The dispersion of microbubbles and nucleating bubbles exfoliate tissue layers as the bubbles rise to the surface of the tank/bath.

Alternately, a smaller subregion, rather than the animal's whole flank, etc., may be treated hereby. In that sense, the region of exposure is subjected to a partial tank or other containment immersion. Depending on the size and shape of said region, it may be possible to treat the animal's leg or legs in a common exposure tank.

In other variations, the treatment region may be exposed to a circulating external bandage, cuff or wrap for surrounding the targeted region. Finally, if only topical treatment is needed (or desired) albeit on a more frequent basis and/or for greater lengths of total exposure time(s), the animal may be subjected to oxygenation treatments by washing/rinsing, showering and/or shampooing in an oxygen-rich solution made per the present invention but then stored in suitable gas-retaining bottles for eventual implementation via washing, rubbing and/or massaging into the treatment regions/zones for any given beast.

One preferred application for this treatment is for those animals that “perform athletically”. More particularly, for racing horses and dogs, this invention achieves improved performance without running afoul of drug-based doping concerns. Furthermore, for any such animal subject to performances (including racing), this invention should achieve better and faster recoveries from injuries to skin and/or muscles.

It should be noted that O₂ is a molecule (O:O) with shared electrons and that O⁻ is an ion (free radical) with an unpaired electron. Water is formed from free radicals: H⁺+(OH)⁻=H₂O. Free radicals are usually reactive oxygen free radicals which can form from, for example, hydrogen peroxide: H₂O₂→H₂O⁻ or ozone: O₃=O₂+O⁻.

This method can also be practiced with any solute/dispersion gas and liquid solvent that is provides benefit to living tissue and/or the host. It may be beneficial, for example to use carbon dioxide as a gas phase to help exfoliate necrotic skin. Carbon dioxide is desirable in this case because of a substantially higher solubility in water as compared to oxygen. During the nucleation of the gas phase to form microbubbles, a significantly greater volume of carbon dioxide gas would be available to form microbubbles as compared to oxygen since dissolved gas is the source of the dispersed gas.microbubbles.

In this instance, the clear solution containing carbon dioxide and water will be first allowed to infiltrate the necrotic skin. Once infiltrated, the solution would be energized using thermal or mechanical energy to cause gas to nucleate and precipitate from solution. In the case of carbon dioxide, a volume change greater than 25:1 will occur. Such an expansion is an energy source for exfoliating necrotic skin that has been infiltrated by the original solution.

### BRIEF DESCRIPTION OF DRAWINGS

FIG. 1 is a cross-sectional view of a two-phase mixture containing a gas enriched solution and microbubble dispersion in accordance with the present invention;

FIG. 2 is a graph plotting bubble/microbubble size versus surface area;

FIG. 3 is a graph plotting various pressure and/or temperature changes to different sized microbubbles;

FIG. 4 is a graph plotting the effect of bubble diameter on terminal velocity;

FIG. 5 is a schematic depicting one method for generating and using a gas-enriched solution and microbubble dispersion per the invention;

FIG. 6 is a schematic of a first alternate method variation;

FIG. 7 is a graph comparing motor power versus impeller RPM;

FIG. 8 is a cross-sectional view of one embodiment of phase contactor according to the invention;

FIG. 9 is a schematic of a first alternate phase contactor embodiment;

FIG. 10 is a perspective view of the shaft to the phase contactor of FIG. 9;

FIG. 11 is a perspective view of the rotator from FIG. 9;

FIG. 12 is a cross-sectional view taken along line A-A of FIG. 9;

FIG. 13 is a graph showing concentration versus pressure for saturated oxygen in solution;

FIG. 14 is a schematic of a second alternate method variation;

FIG. 15 is a flowchart depicting one method for improving animal health and wellness per this invention; and

FIG. 16 is a flowchart showing another method variation with optional steps inserted via dotted lines.

### DESCRIPTION OF PREFERRED EMBODIMENTS

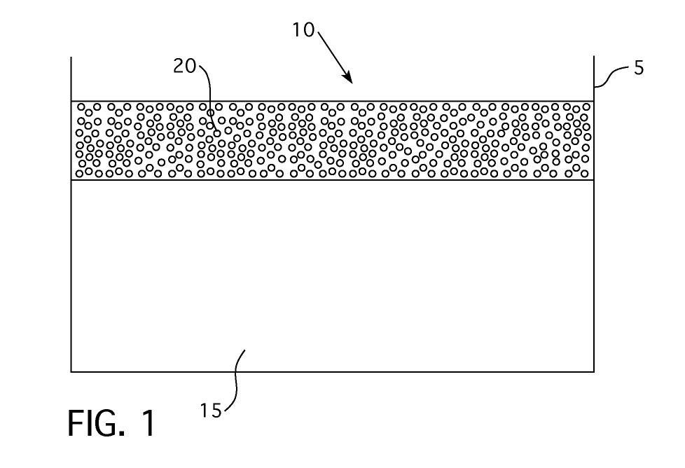

Referring specifically to FIG. 1, a two-phase mixture 10 containing a dissolved gas is illustrated. The mixture 10 contains a homogeneous solution 15 and a suspension or emulsion 20. The solution 15 contains a gas, such as molecular oxygen, dissolved in a solvent, such as water. The suspension 20 is formed by a dispersion of microbubbles containing a gas, such as molecular oxygen. For purposes of this description, the mixture 10 will be described as containing pure molecular oxygen gas in water. However, it is intended that the mixture may contain other solute gases and solvents, as will be discussed further below.

FIG. 1 shows the two-phase mixture in a static condition, where the mixture is stored in a vessel 5. The microbubble dispersion 20 consists primarily of molecular oxygen gas bubbles that have nucleated out of the solution 15. The microbubble suspension 20 has a lower density than the solution phase 15 and therefore forms a stratified layer on top of the solution. Although it is not clear from FIG. 1, the microbubble dispersion 20 typically has an occluded or cloudy appearance believed to be caused by the scattering of visible light energy through the microbubble surfaces.

Referring again to FIG. 1, the homogeneous solution 15 will be described in further detail. The solubility limit of oxygen in water under equilibrium conditions with air (p_{O2}=0.21) at 77° F. is approximately 8.3 mg/l. When the two-phase mixture 10 is initially exposed to atmospheric conditions, the homogeneous solution 15 has a supersaturated or hypersaturated molecular oxygen content, i.e., above the solubility limit of oxygen in water under atmospheric conditions. Preferably, the homogeneous solution 15 has a dissolved oxygen concentration above 20 mg/l at 1 atm, and 65° F. or higher. More preferably, the solution 15 has a dissolved oxygen concentration above about 40 mg/l at 1 atm and 65° F. or higher. As a result, the oxygen concentration in the solution 15 is not stable when exposed to atmospheric conditions. Over time, exposure of the solution 15 to atmospheric conditions will cause some of the dissolved molecular oxygen to be lost through ebullition. More specifically, over time, dissolved oxygen molecules will gradually nucleate out of solution 15 into gas bubbles. Depending on pressure and temperature conditions, the concentration of dissolved oxygen will decrease down to the equilibrium concentration over a period of several minutes.

The supersaturated or hypersaturated molecular oxygen content in solution 15 is preserved by limiting agitation and preventing flow conditions in the solution that can facilitate ebullition of oxygen gases. The high dissolved oxygen content is also maintained by storing the solution 15 in a manner that limits or prevents desorption of the gas. For instance, the solution may be stored and distributed in sealed screw top containers constructed of glass or alternative materials impervious to oxygen diffusion at these high oxygen concentrations.

If oxygenated water is stored in capped bottles, made of an oxygen impervious material, elevated oxygen concentrations can be preserved for extended periods. In an experiment, seven glass bottles were filled with oxygenated water, processed as previously described, and immediately capped. A polargraphic probe was used to measure dissolved oxygen. The initial oxygen concentration was 64.2 mg/l, at a temperature of 17.6° C. Each bottle was uncapped for measurement of oxygen concentration at the intervals below:

| Initial | 6 hours | 1 day | 2 days | 3 days | 4 days |
|---|---|---|---|---|---|
| 64.2 mg/l | 65.7 mg/l | 63.5 mg/l | 67.5 mg/l | 58.5 mg/l | 55.4 mg/l |

It can be seen that over 86 percent of the original dissolved oxygen concentration was retained after 4 days. Such retention of oxygen in solution provides benefits in a number of applications.

As stated earlier, gas microbubbles that nucleate from solution, where the solution is a Newtonian fluid, such as water, rise to the surface and are released into the air above the solution. Gas bubbles rise in such fluids because a net body force exists that projects the bubbles upward. Since Newtonian fluids yield to these forces, the bubbles rise. The mechanics that control bubble rise are explained by Stokes Law, which will be examined later. In some applications, it is desirable to limit or substantially prevent bubbles from rising to the surface of the solution during storage and to maintain the micro-bubble dispersion indefinitely. In particular, it may be commercially desirable to market a product that contains visible oxygen bubbles held indefinitely in a suspension.

A supersaturated or hypersaturated solution of molecular oxygen in water is unstable at ambient pressure by definition. If, for example, the ambient temperature and pressure conditions establish an equilibrium oxygen concentration of 8 mg/l, and an oxygenated solution containing 40 mg/l is prepared at 5 atmospheres pressure, such a solution will have an oxygen concentration of 32 mg/l above the solubility limit. The oxygen-water system will attempt to reject oxygen by nucleating oxygen bubbles. Nucleation can be either a homogeneous or heterogeneous process depending on changes in temperature, mechanical agitation, or the presence of suitable particles that can stimulate gas nucleation. Rapid pressure changes can provoke gas bubble nucleation, and in this invention, a reduction of pressure to ambient typically results in the formation of microbubbles.

The microbubble dispersion 20 is characterized as having a very large surface area through which interfacial transport of oxygen occurs. Interfacial transport of molecular oxygen through a large surface area aids in resupplying oxygen to solution when dissolved oxygen is taken up during chemical reactions. As a result, a large surface area in the microbubble dispersion is desirable.

The mixture 10 preferably contains microbubbles having an average bubble diameter of about 2-9 μm or 10-100 microns. Microbubbles in this size range provide a significantly larger surface area than a cluster of large bubbles containing the same volume of gas. The magnitude of this difference can be visualized by performing calculations for several bubble diameters at a constant volume of gas. The following calculations show the surface areas present for a single bubble, a plurality of one-inch diameter bubbles and a plurality of 50-micron or 5 μm diameter microbubbles, wherein each calculation is based on one cubic foot of gas. The value, r, is the radius of a single bubble, Vₒ is the volume of a single bubble, Aₒ is the surface area of a single bubble, and A is the aggregate surface area for the bubble formation:

a. Single bubble:

Vₒ=4/3πr³; r=(3Vₒ/4π)^{1/3}

Thus, when Vₒ=1.00 ft³, r=0.62 ft. Therefore, the diameter of a single bubble containing 1.00 ft³ of gas=1.24 ft.

The surface area of this single bubble (A_{b}) is:

A_{b}=4πr²=4π(0.62 ft)²=4.83 ft²

b: For one inch bubbles:

r=0.50 inches=0.042 ft.
The volume of a single bubble (V_{b}) is:

V_{b}=4/3πr³=4/3π(0.042 ft)³=3.1×10⁻⁴ ft³/bubble

A_{b}=4πr²=4(0.042 ft)²=2.22×10⁻² ft²

The number of one inch bubbles (n_{b}) in a 1.00 ft³ volume of gas is:

n_{b}=Vₒ/V_{b}=1.00 ft³/3.1×10⁻⁴ ft³/bubble=3,224 bubbles

The surface area (Aₒ) of a 1.00 ft³ volume of gas comprised of one inch bubbles therefore is:

Aₒ=ΣA_{b}=n_{b}A_{b}=3,224 (2.22×10⁻² ft)=71.43 ft²

c. For 50μ microbubbles:

r=25μ/3.05×10⁵ μ/ft=8.2×10⁻⁵ ft

V_{b}=4/3πr³=4/3π(8.2×10⁻⁵ ft)³=2.31×10⁻¹² ft³

A_{b}=4πr²=4π(8.2×10⁻⁵ ft)²=8.45×10⁻⁸ ft²

n_{b}=Vₒ/V_{b}=1.00 ft³/2.31×10¹² ft³=4.32×10¹¹ bubbles

Aₒ=n_{b}A_{b}=4.32×10¹¹(8.45×10⁻⁸ ft²)=36,504 ft²

d. For 5 μm microbubbles:

r=2.5μ/3.05×10⁵ μ/ft=8.2×10⁻⁶ ft

V_{b}=4/3πr3=4/3(8.2×10⁻⁶ ft)³=2.31×10⁻¹⁵ ft³

A_{b}=4πr²=4(8.2×10⁻⁶ ft)²=8.45×10⁻¹⁰ ft²

n_{b}=Vₒ/V_{b}=1.00 ft³/2.31×10⁻¹⁵ ft³=4.32×10¹⁴ bubbles

Aₒ=n_{b}A_{b}=4.32×10¹⁴(8.45×10⁻¹⁰ ft²)=365,800 ft²

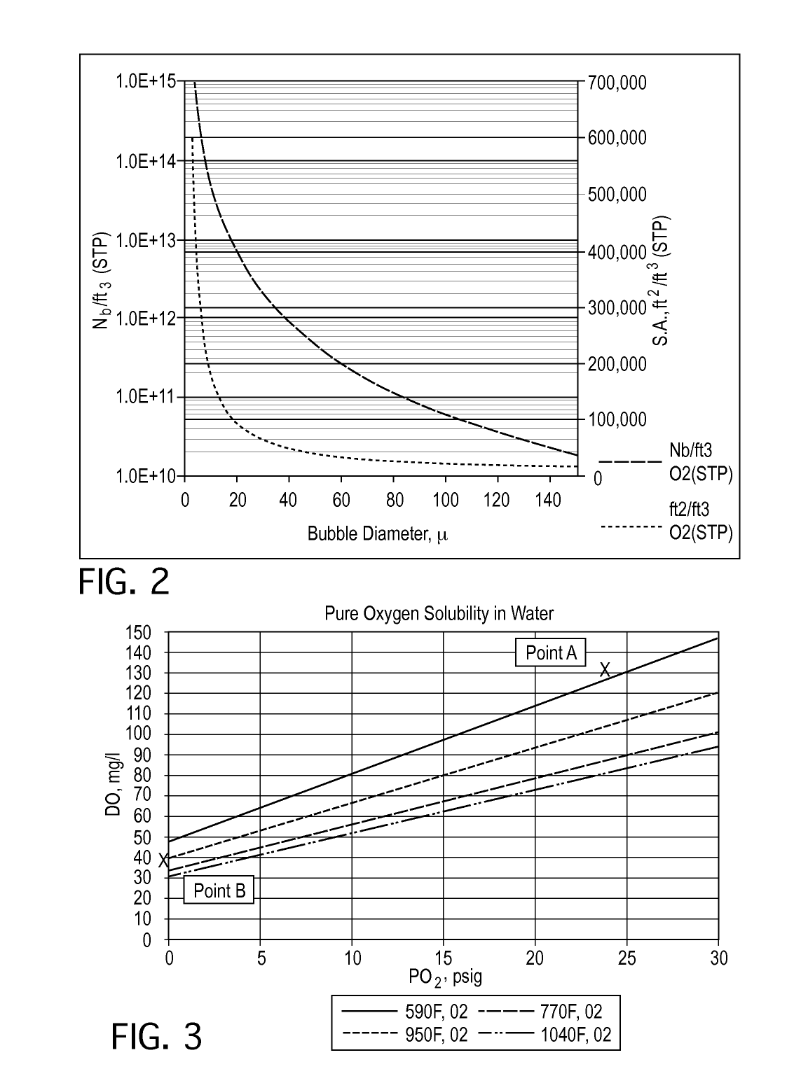

These calculations are summarized in accompanying FIG. 2.

The driving force for microbubble formation is shown in accompanying FIG. 3. At 25 psig and 59° F., the solubility of pure oxygen in water is 130 mg/l, as depicted by “Point A”. That solubility of pure oxygen in water decreases to about 30 mg/l at 0 psig (ambient pressure) and 104° F. (Point B). Approximately 100 mg oxygen per liter of water is rejected from liquid solution and available to form microbubbles if an oxygenated solution at Point A is exposed to the temperature and pressure conditions of Point B. At Standard Temperature and Pressure (or “STP”), 100 mg of pure oxygen occupies a volume of 71 ml. Even with this relatively modest difference in pressure and temperature conditions, a pure oxygen gas fraction of 6.6% is attainable from nucleated dissolved oxygen. An STP oxygen volume of 128 ml is equivalent to 0.0025 ft³. Based on FIG. 2, such a volume of gas in the form of 20 micron microbubbles will create 245 ft² of surface area and represent 23.2×10⁹ microbubbles. These microbubbles will provide a pure oxygen environment to surfaces in contact with them, and the remaining dissolved oxygen concentration in water of 30 mg/l has an equivalent oxygen pressure of 0.75 atmospheres.

Based on the foregoing calculations, the aggregate surface area for a dispersion of gas bubbles increases by a factor of 10 as the radius of the bubbles decreases by a factor of 10. Referring to calculations (b) and (c), and within rounding error, a dispersion of 50-micron diameter bubbles containing one cubic foot of gas will have an aggregate surface area more than 500 times greater than a dispersion of one-inch bubbles containing the same volume of gas; and a dispersion of 5 μm diameter bubbles containing one cubic foot of gas will have an aggregate surface area 10 times greater than the 50-micron diameter bubbles and 5,120 times greater than a dispersion of one-inch bubbles containing the same volume of gas.

The microbubble suspension 20 is somewhat unstable, as the microbubbles will rise to the surface of the mixture and pass into the atmosphere over time. This movement is generally driven by buoyancy (body) forces. The mechanics of microbubble separation in a liquid can be analytically described for by Stokes' Law for small bubble sizes. Particularly:

V=2gr²(ρ_{g}−ρ_{w})/9_{η},

where V is the terminal velocity of a bubble rising through the liquid, g is the acceleration of gravity, r is the radius of the bubble, ρ_{g} is the density of the gas, ρ_{w} is the density of the liquid, and η is the Newtonian viscosity of the liquid.
Based on the formula, the terminal velocity of a rising bubble is proportional to the square of the radius of the bubble. In other words, the net upward force that causes a bubble to rise (i.e., the buoyancy force less all drag forces on the bubble) increases dramatically as the size of that bubble increases.

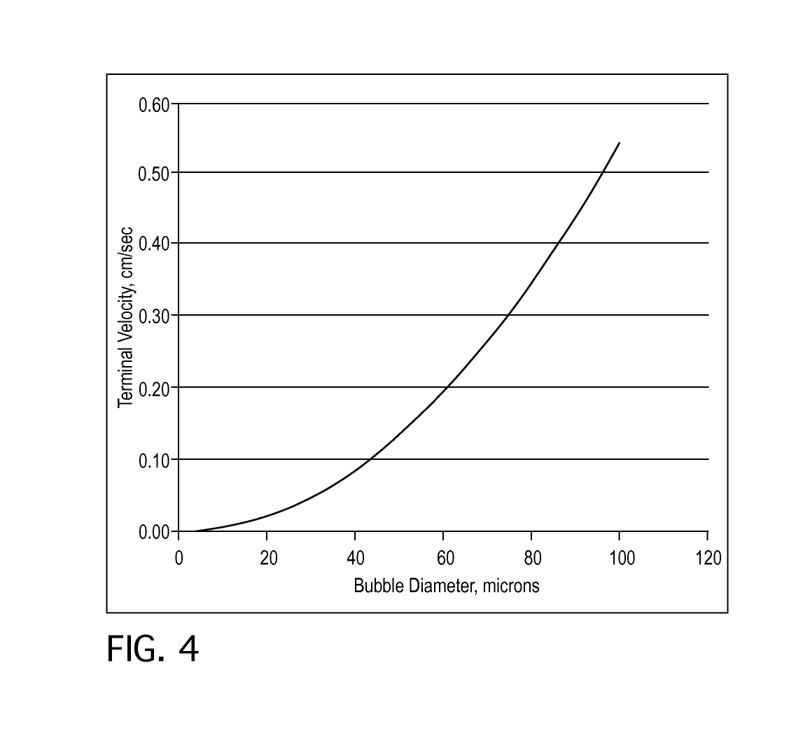

This effect is graphically illustrated in FIG. 4. Therein, terminal velocity values for bubbles in the forgoing system have been calculated as a function of bubble diameter. A 20 micron diameter bubble, for example, has an associated terminal velocity of approximately 0.3 cm/sec. Absent the effects of any circulation flow in a water bath, such bubbles formed at the bottom of a 24 inch (61 cm) deep bath will have a residence time of over 30 minutes; and microbubbles of 50 microns in diameter will have a residence time over 6 minutes. For this reason, it is advantageous to minimize bubble size so that the rate of bubble rise is also minimized.

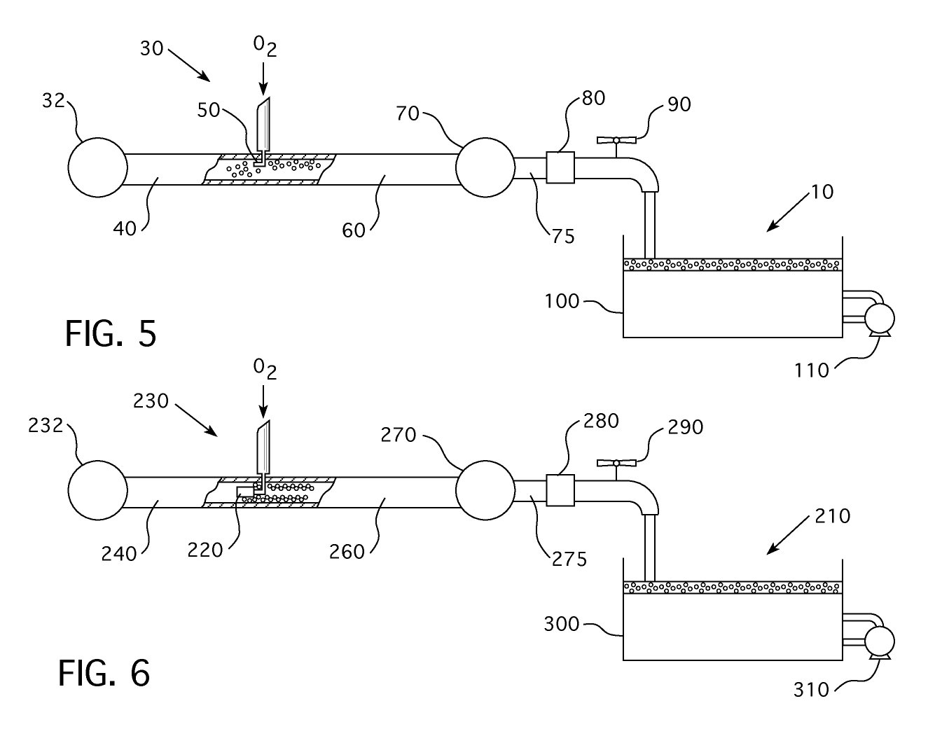

Referring now to FIG. 5, a method for using a two-phase oxygenated mixture 10 in a bath 100 is schematically illustrated. Water having a desired temperature is pumped through an oxygenation system 30. More specifically, water is conveyed through a pre-charge pump 32 to pressurize said water. Preferably, the pressure of the stream is between 35 to 450 psig, more preferably around 120 to 150 psig. In addition, the water preferably has a temperature no greater than 65° F., as warmer temperatures decrease the solubility of the gas in solution and may not be appropriate for the medical condition being treated. The water is discharged from the pre-charge pump 32 and conveyed to the point of gas introduction, or phase contactor 30, through an influent line 40 maintained at low pressure. Oxygen-containing gas is introduced into the influent line 40 from a supply of gas.

In FIG. 5, oxygen gas is shown being injected into a liquid stream through nozzle 50, which comprises the phase contactor. That gas is injected substantially countercurrent to the flow direction in the influent line 40 at a high velocity. Countercurrent injection of the gas facilitates a more complete mixing of the gas in solution, as a result of the instability of the jet plume. Injecting the gas at relatively high velocities further enhances mixing. Preferably, the gas is injected into influent line 40 at a pressure between about 150 to 450 psig. (Note that: in addition to oxygen, feed gas can comprise at least one gas selected from the group consisting of air, molecular oxygen, hydrogen and nitrogen, atomic argon, helium, and neon.)

The elevated pressure substantially limits any remaining gas bubbles from increasing in size. The amount of pressure in discharge line 75 varies depending on the size of the system and desired discharge conditions. Preferably, the pressure of the mixture as it enters the discharge line 75 is between about 35 and 450 psig. In the case of oxygen, the amount of this gas that is dissolved in water increases with increasing pressure, as per FIG. 3. The high-pressure stream is conveyed to a spigot 90 where it is discharged into bath 100. Alternately, depending on the pressure head in the high-pressure stream, the liquid stream may be conveyed through a pressure reducer 80 prior to being conveyed to spigot 90. The dissolved oxygen content in the liquid 10 at the point of discharge can be as high as 200 mg/l and the liquid is substantially free of gas bubbles since the gas is substantially in solution.

As liquid 10 discharges into bath 100, the tank is allowed to fill with minimal agitation or stirring so as to substantially minimize the amount of nucleation and ebullition of gas bubbles. In one preferred embodiment, liquid 10 is expanded through a nozzle or orifice while entering bath 100. The resultant abrupt pressure decrease causes an instantaneous change in gas solubility. That, in turn, causes some precipitation of the dissolved gas to form bubbles of substantially pure gas. If this expansion occurs with sufficient shear, the resulting bubble population will be microbubbles.

Additionally, a secondary stream of liquid at a temperature greater than liquid 10 is mixed with same. As a result, the temperature of the combined liquids increases. That temperature rise also decreases the solubility of gas in liquid 10 and produces bubbles of substantially pure gas.

It has been found that if the two liquids are mixed in a coaxial manner, the resulting bubble population consists substantially of microbubbles and appears almost smoke-like within bath 100. In this way, the maximum quality of gas originally dissolved in liquid 10 is converted to a dispersion of microbubbles within bath 100. In the case of oxygen in water, the bath 100 is preferably filled so that the dissolved oxygen concentration is maintained somewhat above 20 mg/l at 1 atm and 65° F.

Once bath 100 is filled, the solution is allowed to interact with animal tissue. Some of the dissolved oxygen contacts the tissue where it is believed to assist with the regeneration of new tissue cells and also be transported through the skin and enter the bloodstream through capillaries. Additionally, microbubbles of oxygen contact the skin and are expected to provide at least an equivalent benefit to the skin being exposed to pure oxygen in an oxygen tent. The very small diameter of the microbubbles results in an extended retention time within bath 100.

Although elevated dissolved oxygen content in the bath 100 is not stable under atmospheric conditions, in the absence of bubble nucleation, the rate of oxygen liberation at the liquid/atmosphere interface is slow enough that the dissolved oxygen content therein can remain elevated for several hours. After some time, the dissolved oxygen content will decrease down to equilibrium conditions. Preferably, ambient pressure at the location of the bath is maintained between 0.9 atm and 1.1 atm.

Additional energy may be added to the bath solution to stimulate the nucleation of microbubbles and accelerate the exfoliation process. Mechanical mixing or circulation of the bath solution using stirring bars, circulation pumps or other mechanical devices may also stimulate nucleation of microbubbles. In addition to the foregoing, still other stimulation enhancements may include adding one or more localized ultrasonic transducers to the system.

Ordinarily, liquid 10 in continuously introduced into bath 100 resulting in outflow of liquid into a drain. Such one-pass systems are desirable for maximizing sanitation in situations with multiple users. A pipe placed within bath 100 establishes the desired bath depth. It would also allow discharge liquid to flow into a drain for disposal.

As an alternative to a one-pass system, liquid 10 can be recirculated. In FIG. 5, a circulation pump 110 is shown which gently draws solution from the bath and recirculates same back into the bath for certain applications where it is desirable to do so. In some cases, heat dissipation from the submerged tissue may be sufficient to promote nucleation of microbubbles in the proximity of the tissue. Moreover, the addition of solid surfaces in the bath may be used to stimulate heterogeneous nucleation of microbubbles.

The generation of smaller bubbles in the liquid stream for dissolution requires a significant amount of energy. One method of accomplishing this is to introduce gas into the liquid at a very high speed. In the present method, the gas is preferably introduced at supersonic conditions at the exit of the nozzle 50. The nozzle 50 may be any type of nozzle that permits supersonic gas flow conditions, such as the nozzle disclosed in U.S. Pat. No. 5,463,176. The velocity of the gas at the exit of the nozzle 50 is preferably in the range of Mach 1 to Mach 5 and more preferably in the range of Mach 2 to Mach 4. It will be understood that lesser velocities, such as those below Mach 1, can be used but ordinarily will not provide as much mixing of gas into solution.

The introduction of gas at supersonic conditions into the low-pressure stream creates a two-phase gas/liquid oxygenated mixture 10. The mixture 10 is conveyed through an axial flow turbine based pump known as a co-compressor 70, which concurrently increases the pressure of both gas and liquid in the stream and discharges the mixture into a high-pressure discharge line 75. The pressures of the gas and liquid are increased to allow large quantities of oxygen to efficiently dissolve into the liquid in a short period of time.

Multiple stage axial flow pumps have been found to be suitable for increasing pressure. Diaphragm or piston pumps may be even more suitable because rotational flow is not present that can result in density separation of the gas and liquid, however the flow capacities of such pumps are categorically lower than axial pumps.

As noted, common methods for oxygenating water include using gas diffusers at atmospheric and elevated pressures, packed beds and pressure tanks. Pressurized oxygen gas may be introduced through a submerged pipe having small holes or orifices into a vessel of water. Gas pressure is sufficient to overcome the hydrostatic head pressure while sustaining pressure losses during passage through the small gas orifices. As a result, bubble aeration occurs at relatively low pressures; this pressure being predominantly a function of tube immersion depth. The decrease in gas pressure across an orifice results in an increase in gas kinetic energy. This kinetic energy satisfies the energetic requirement to create surface area, albeit at a low level in this case. Since all interphase interfaces have a characteristic surface energy, the creation of interfacial (surface) area is an energetic process.

Area and velocity are inversely proportional. Hence, as the orifice diameter decreases, the corresponding pressure drop and gas velocity increase, and more surface area is generated. Smaller bubbles result.

This method has a limiting condition in that the amount of heat (as irreversible work) produced is inversely proportional to the square of orifice diameter. It therefore becomes impractical and energetically inefficient to operate at exceptionally small orifice diameters. This process also has an absolute limit as a gas velocity of Mach 1 is approached within the pore. Because a pore lacks the convergent/divergent geometry required to achieve supersonic flow, increasing pressure beyond the critical pressure will not result in a further reduction of bubble size. As noted earlier, a dispersion of very small bubbles, e.g., bubbles having diameters of about 50 microns, will have a much larger total surface area than a dispersion of large bubbles occupying the same volume.

One process uses a Mach 3 supersonic nozzle to dissipate energy at significantly higher levels than conventional orifice-based oxygenation technologies. Oxygen issues from the nozzle countercurrent to water flow, resulting in the explosion of the jet plume into a quasi-emulsion.

A first schematic method alternative is shown in FIG. 6 wherein components similar or identical to those in FIG. 5 are identified by a similar reference number in the next hundred series. In FIG. 6, a two-phase oxygenated mixture 210 is illustrated. Water having a desired temperature is pumped through oxygenation system 230. The water is conveyed through a pre-charge pump 232 to pressurize it. Preferably, the pressure of the stream is between 35 psig to 120 psig. In addition, the water has a preferred temperature no greater than 65° F., as warmer temperatures decrease solubility of the gas in solution and may not be appropriate for its intended use.

The water discharged from pre-charge pump 232 is conveyed to the oxygenation system 230 through an influent line 240 maintained at low pressure. Oxygen-containing gas is introduced into influent line 240 through a porous diffusion device 220 connected to a supply of oxygen gas. The diffusion device 220 may have various geometries and be placed in a variety of ways to contact with the liquid.

In FIG. 6, a cylindrical diffusion device 220 is shown disposed inside the influent line 240. Gas is delivered through the diffusion device 220 and into the pressurized liquid through a plurality of pores disposed through the cylindrical face of the diffusion device. Preferably, the pores are no larger than 2 microns in diameter to facilitate the formation of small bubbles of the gas. The pressurized liquid flows past the diffusion device 220 in a direction transverse to the axis of the pores on the diffusion device 220 to create shear stresses along the outlets of the pores. As such, the shear stresses overcome attachment forces and surface tension that hold the small gas bubbles on the diffuser to detach and transport these bubbles as soon as they are formed on the diffuser face. In this way, the coalescence of large bubbles on the surface of the diffuser 220 is minimized allowing the small bubbles with high surface area to rapidly dissolve in the liquid.

As noted earlier, introducing molecular oxygen to water through a porous diffuser results in relatively large bubbles with low surface area. Increasing gas pressure to provide an increased flow through the pores results in smaller bubbles. If a nozzle having convergent/divergent geometry is used, an increased gas velocity is achieved due to the conversion of pressure energy to surface energy with even smaller bubbles resulting in increased bubble surface area and greater dissolution. Greater surface area of bubbles results in higher levels of dissolved oxygen in water. (Note that: a convergent/divergent nozzle is capable of supersonic gas flow.)

It has been found that limitations exist on the amount of gas/liquid surface area that can be generated by gas expansion through porous materials or nozzles. In one preferred embodiment of this invention, an improved phase contactor was created consisting of a rotating gear like device known as an impeller used to create turbulence and eddys within the incoming water stream. Gas (in this case, oxygen) is introduced to a region in the vicinity of the rotating impeller. That gas stream instantly interacts with the turbulent eddys created by the rotating impeller with the resulting shear believed to reduce the gas stream into very small bubbles. The energy necessary to create gas/liquid interfacial area is supplied by the rotating impeller and not a diffuser or nozzle as previously described.

The rotating impeller will: a) create shear within the body of liquid to form gas bubbles; b) disperse gas bubbles throughout the liquid; and c) agitate the liquid. Efficient bubble formation is dependent on maximizing shear forces in the fluid phase. Energy (i.e., ergs/cm³) is required to generate gas/liquid interfacial area. This energy may be supplied by the reaction of non-rotating liquid with the impeller (shear). Shear is maximized when the radial velocity gradient in the liquid is as great as possible. Alternatively, maximum shear is produced when the radial velocity differential between the liquid phase and rotating impeller is maximized.

Newton's Law of viscosity describes the foregoing situation, viz:

τ=−η(dv_{φ}/dr),

where: τ=shear force, η=Newtonian viscosity (laminar) and dv_{φ}/dr=radial velocity gradient. The radial velocity gradient, dv_{φ}/dr, must be maximized to produce the highest values of τ.

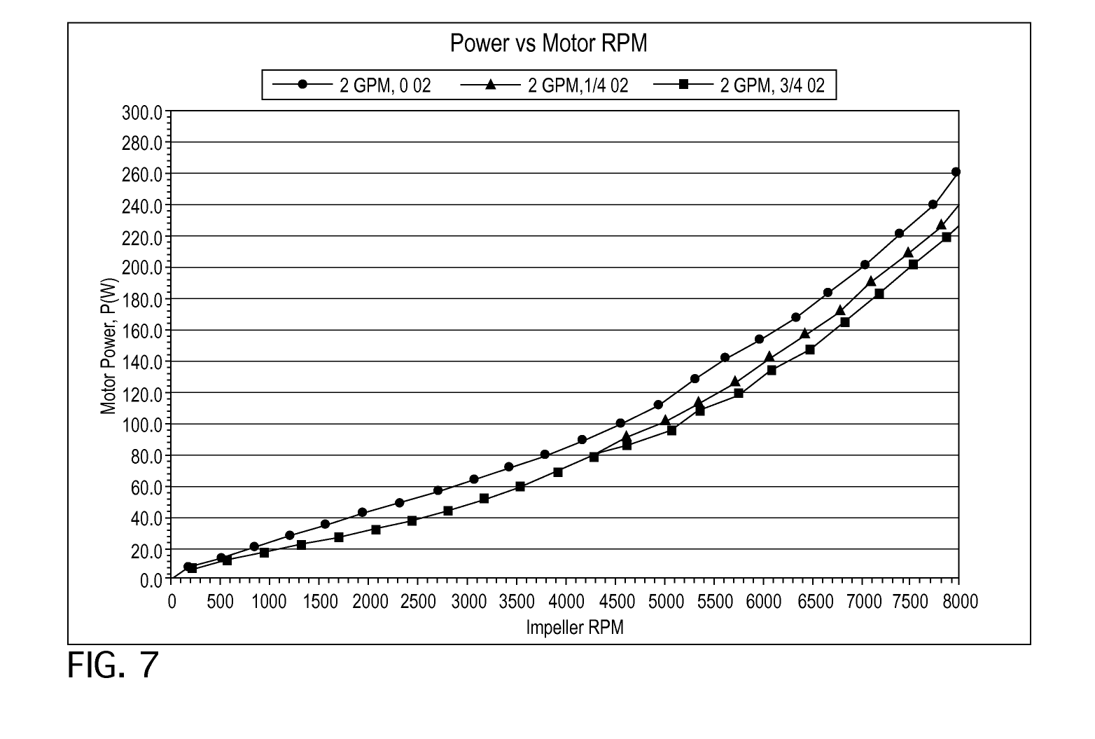

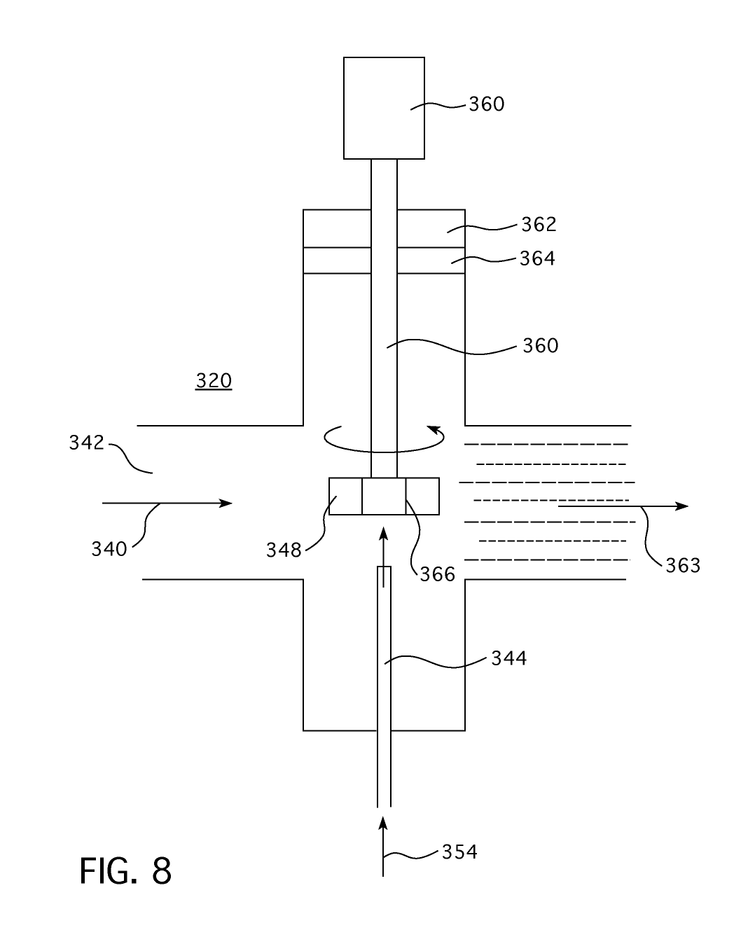

In the case of the rotating impeller, power input increases as the cube of impeller speed. FIG. 7 demonstrates this effect in the case of water and two flow rates of oxygen. One representative impeller system for accomplishing preferred flow rates is shown in accompanying FIG. 8. Therein, liquid 340 enters the body of the phase contactor 320 through conduit 342. Drive motor 360 rotates shaft 350 supported by bearings 362 and 364 with an attached impeller 348 with local radial projections 366. As impeller 348 rotates within liquid 340, turbulent eddys are created behind the trailing edges of radial projections 366. These eddys interact with gas 354 that is introduced through tube 344. This interaction results in a reduction of the stream consisting of gas 354 into small bubbles of high surface area. The movement of liquid 340 through the body of phase contactor 320 displaces the bubbles and produces liquid/gas outflow 363.

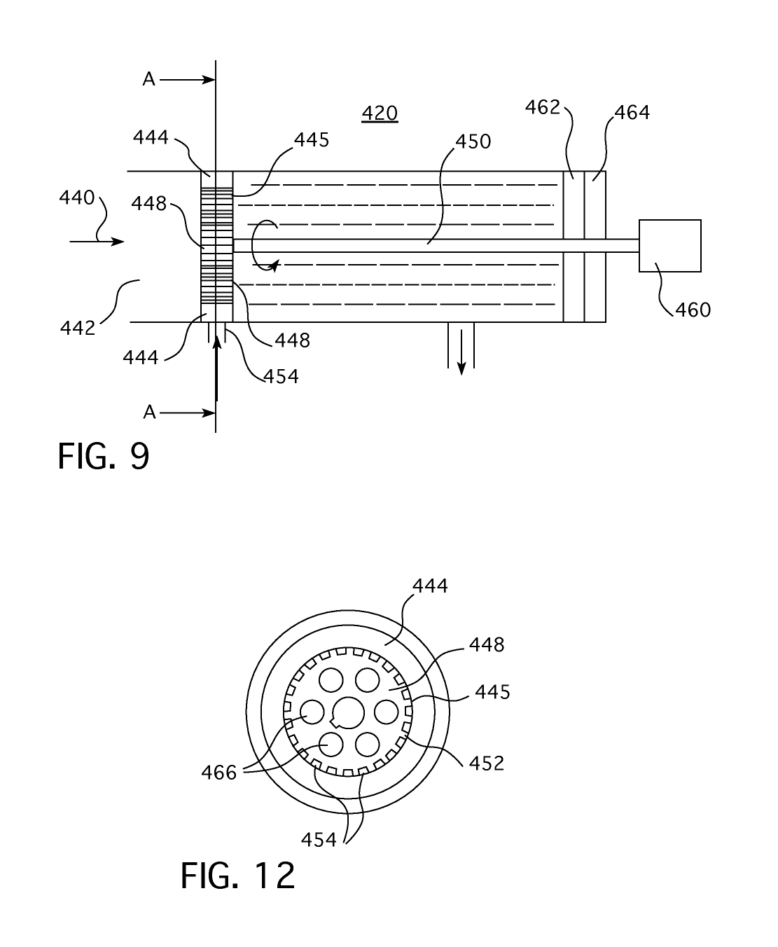

In FIGS. 9-12, an alternate version of phase contactor 420 is shown that is capable of producing bubbles in the range of 5 to 250 μm diameter. For FIGS. 9-12, components common with those depicted in earlier FIG. 8 are similarly numbered, though in the 400 Series. Therein, water 440 is introduced to phase contactor 420 at end 442 and gas is introduced through ring diffuser 444. The gas is mixed with and dissolved in the water to provide a mixture of solution having gas dissolved therein and bubbles of gas. A rotator 448, which is turned by shaft 450, is mounted to turn within inside surface 445 of diffuser 444. Rotator 448 is mounted within diffuser 444 to provide preferably a zero clearance fit, and yet the rotator should be permitted to rotate relatively freely. Rotator 448 is provided with teeth 452 which extend across width 450 of the diffuser. These teeth are separated by grooves 454.

Diffuser 444 is comprised of a porous material to provide pores in a size range of 0.1 to 50 microns (broad size range, narrow size range). Gas is introduced through valve 454 and flows circumferentially through diffuser 444 and then radially inwardly to form gas bubbles in the liquid, particularly in grooves 454. As the rotator spins, it provides a shearing action with the result that bubbles forming are divided into much smaller bubbles, thereby increasing the surface area of the bubbles and greatly increasing dissolution of the gas in liquid.

Diffuser ring 444 may be formed from any suitable material that permits the flow of gas there through. Suitable materials are materials comprised of porous stainless steel, copper and alloys, nickel alloys, ceramics (Si₃N₄), porous carbon and titanium.

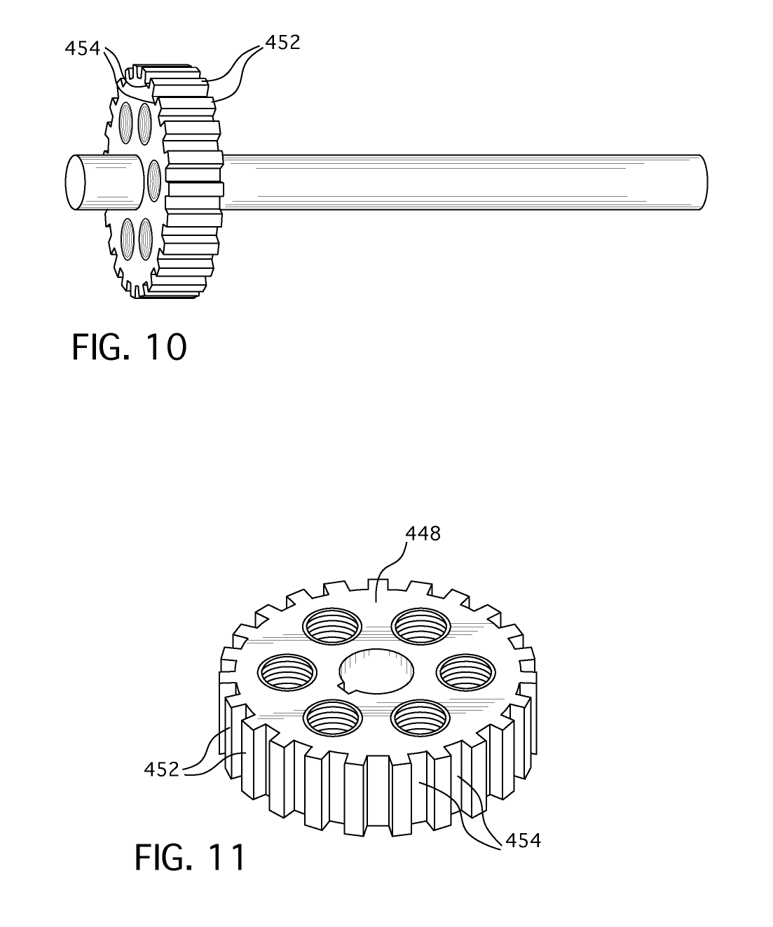

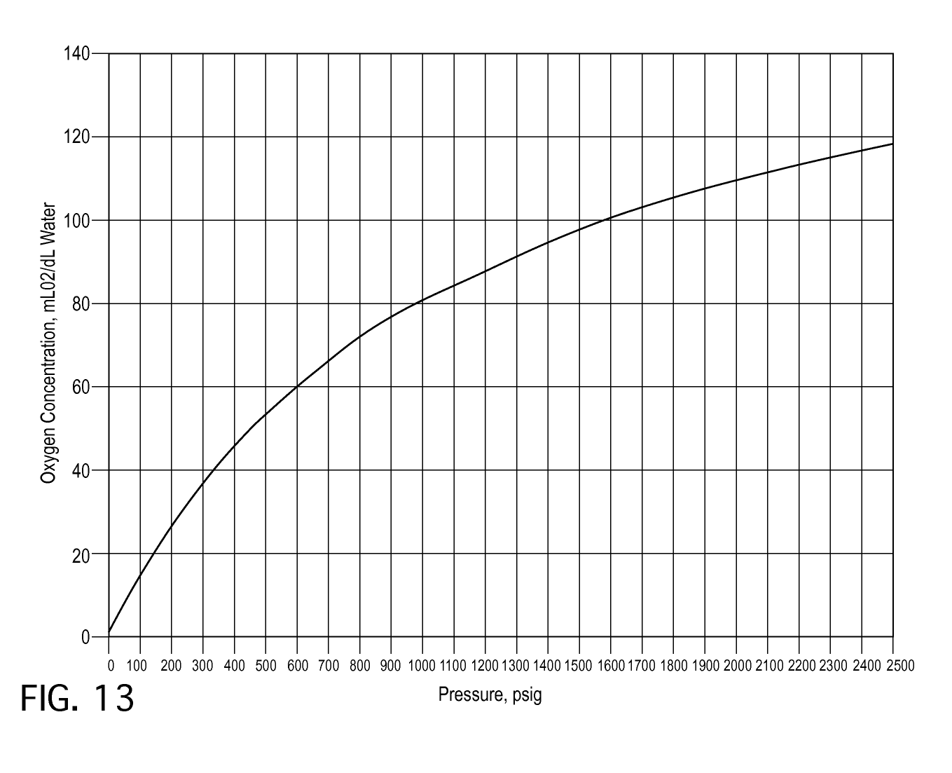

Rotator 448 may be formed from a metal or a plastic material. Preferably, the rotator is formed from stainless steel or Teflon®, titanium, nickel alloys, or ceramic may be used. During operation, the rotator would typically spin at a speed in the range of 75 to 5500 rpm. Shaft 450 may be driven by an electric motor 460. Shaft 450 is mounted on bearing 462 and sealed by end 464. As shown in FIG. 10, rotator 448 is provided with channels 466 which permit additional flow of liquid through the rotator.

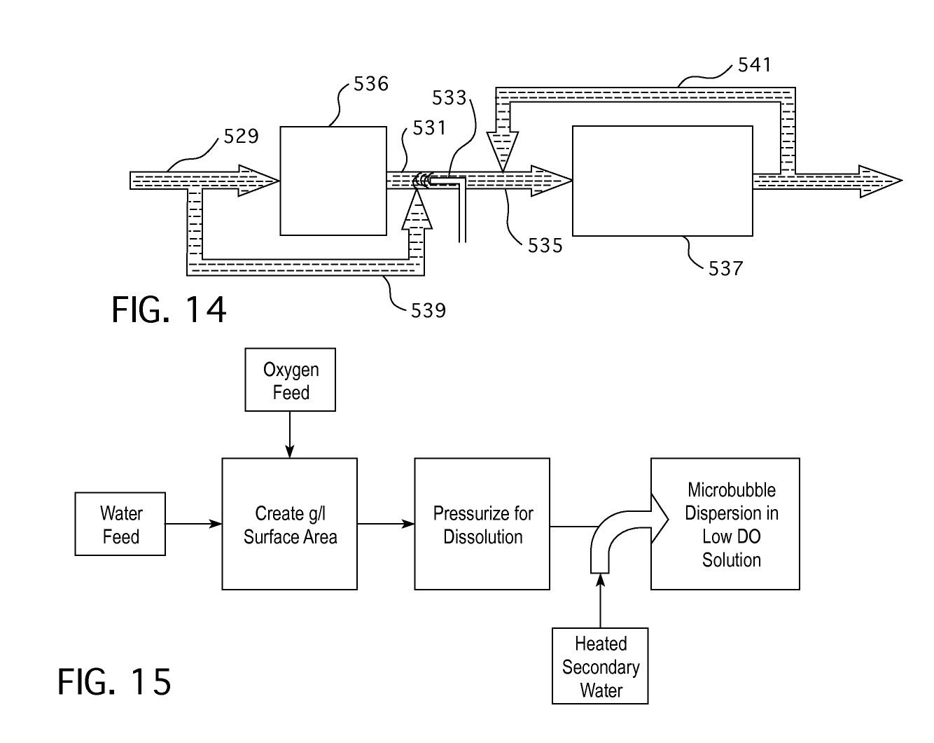

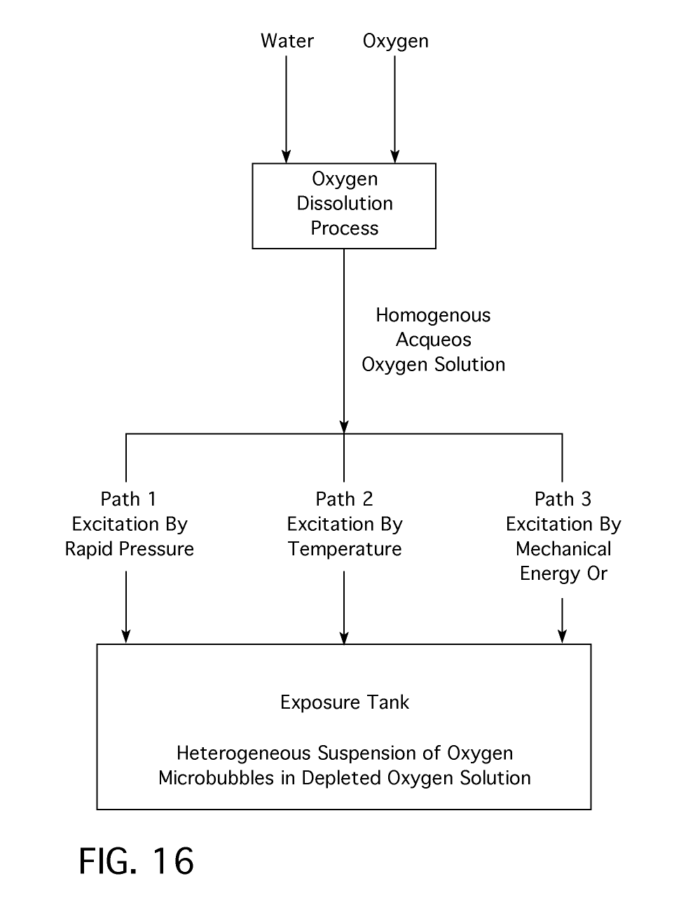

Another schematic method alternative is shown in FIG. 14 wherein elements similar to earlier described components are commonly numbered, though in the 500 Series. Therein, water is introduced along line 529 to low pressure pump 536. That water is pumped to a low pressure, typically about 45 psig. Said water is removed along line 531 where a gas, e.g., O₂, is introduced in a counterflow direction through nozzle 533 to provide a mixture 535 of gas and water. The mixture is then introduced to a high pressure pump 537 where it can be pumped to pressures such as 300 psig or more to provide hypersaturated gas and liquid or oxygen and water which can be used directly or stored. Pump 536 is provided with a bypass 539 and pump 537 is provided with bypass 441.

The terms and expressions, which have been employed herein, are used as terms of description and not of limitation. There is no intention in use of such terms and expressions of excluding any equivalents of the features shown and described or portions thereof. It is recognized that various modifications of the embodiments described herein are possible within the scope and spirit of the invention. While the two-phase oxygenated mixture has been described primarily in terms of its use in skin products and topical treatment, the invention is intended for use in any application where a supply of oxygen, via an oxygen tent, may be desired.

The invention is intended to encompass a wide range of solutes and solvents other than oxygen and water. For instance, injecting nitrogen gas into a solvent can form a two-phase mixture in accord with the present invention. A bath solution may be prepared using one or more gases, including, but not limited to air, carbon dioxide, nitrous oxide or a number of inert gases. Still other treatments may be realized by sequentially treating individuals in multiple gas phases. For instance, an initial treatment with CO₂, followed by O₂ and then, lastly N₂O.

Reference to a range herein is meant to include all the numbers in the range, as if specifically set forth. For example, the range of 5-200 would include numbers 6, 7, 8 . . . 198, 199.

Reference herein to oxygen is meant to include molecular oxygen, but reference to molecular oxygen is meant to include only molecular oxygen or diatomic oxygen, or non-free radical.

Having described the presently preferred embodiments, it is to be understood that the invention may be otherwise embodied within the scope of the appended claims.

What is claimed is:

1. A method of treating animal tissue to revitalize said animal tissue, said method comprising:
(a) dissolving a gas in a liquid to form a solution under elevated pressure for saturating or supersaturating dissolved gas into said solution;
(b) providing a container into which said tissue may be situated for prolonged contact with said solution;
(c) situating said tissue in said container;
(d) introducing said solution through one or more entry points into said container;
and
(e) nucleating gas microbubbles from said solution at or near said entry points and leaving a residual gas that remains in solution, said animal tissue being revitalized through contact with said nucleated gas microbubbles and said residual gas in solution for a predetermined amount of time.

2. The method of claim 1 wherein said solution is introduced into said container at a pressure of about 12 to 325 psig and a one or more temperatures between about 45 and 115° F.

3. The method of claim 1 wherein said tissue revitalizing includes: removing dead or contaminated cells from said animal tissue through contact with said nucleated gas microbubbles.

4. The method of claim 1 wherein said gas is selected from the group consisting of:
oxygen, air, nitrogen, carbon dioxide, argon and combinations thereof.

5. A method of treating animal tissue to revitalize said animal tissue, said method comprising:
(a) dissolving a gas in a liquid to form a solution under elevated pressure for saturating or supersaturating dissolved gas into said solution;
(b) providing a container into which said tissue may be situated for prolonged contact with said solution;
(c) situating said tissue in said container;
(d) introducing said solution with a secondary liquid stream through one or more entry points into said container at a pressure of about 12 to 325 psig; and
(e) provoking a nucleation of gas microbubbles from said solution at or near said entry points and leaving a residual gas that remains in solution, said animal tissue being revitalized through contact with said nucleated gas microbubbles and said residual gas in solution for a predetermined amount of time.

6. The method of claim 5 wherein said gas is selected from the group consisting of:
oxygen, air, nitrogen, carbon dioxide, argon and combinations thereof.

7. The method of claim 5, which further includes mechanically vibrating said solution.

8. The method of claim 5, which further includes adding ultrasonic energy to said solution.

9. The method of claim 5 wherein said solution includes a two phase suspension, said suspension including oxygen microbubbles in a dissolved concentration at or less than its solubility limit at atmospheric pressure.

10. The method of claim 5 wherein said solution contains dissolved oxygen at above about 20 mg/l.

11. The method of claim 10 wherein said solution contains dissolved oxygen at above about 40 mg/l.

12. The method of claim 5, which includes reducing pressure in said container to between about 0.9 atm and 1.1 atm.

13. The method of claim 5 which comprises contacting said tissue with said microbubbles in said solution at one or more temperatures between about 65 and 120° F.

14. The method of claim 13 which comprises contacting said tissue with said microbubbles in said solution at one or more temperatures between about 90 and 115° F.

15. A method for improving animal health and wellness through an aqueous treatment of animal skin, muscle or both, said method comprising:
(a) dissolving oxygen in a liquid to form a solution under elevated pressure for saturating said solution with dissolved oxygen;
(b) providing a container into which at least some portion of animal skin or muscle may be exposed to said solution for a predetermined time; and
(c) introducing said solution through one or more entry points into said container with at least one of:
(i) a heated water stream;
(ii) rapid change in pressure
(iii) mechanical vibration; and
(iv) supplemental ultrasonic energy, said solution introducing provoking a nucleation of oxygen microbubbles at or near said container entry points.

16. The method of claim 15 wherein said solution contains dissolved oxygen at concentrations above about 20 mg/l.

17. The method of claim 16 wherein, in the dissolving step, the solution of dissolved gas is supersaturated and has a dissolved oxygen concentration above 40 mg/l.

18. The method of claim 15 wherein, in the immersing step, areas of animal skin or muscle are immersed into a microbubble-containing solution having a temperature between about 65 and 115° F.

19. The method of claim 15, which includes reducing pressure of said solution and maintaining said pressure between 0.9 atm and 1.1 atm.

20. The method of claim 15, wherein the portion of animal skin or muscle is treated by solution exposure in at least one of the group selected from: a full body tank; a partial, localized bath and a solution-circulating cuff or bandage.
